(19) United States
(12) Patent Application Publication
(10) Pub. No.: US 2025/0333422 A1
(43) Pub. Date: Oct. 30, 2025

(54) AMORPHOUS BERBERINE WITH ENHANCED BIOAVAILABILITY

(71) Applicant: Nature's Way Products, LLC, Green Bay, WI (US)

(72) Inventors: Bob Doster, Oneida, WI (US); Kristen Tisch, Green Bay, WI (US); Scott Brady, Oneida, WI (US); Md Reza, Howard, WI (US)

(73) Assignee: Nature's Way Products, LLC, Green Bay, WI (US)

(21) Appl. No.: 19/192,637

(22) Filed: Apr. 29, 2025

Related U.S. Application Data
(60) Provisional application No. 63/640,054, filed on Apr. 29, 2024.

(51) Int. Cl. C07D 491/147; A61K 31/4355

(52) U.S. Cl. CPC C07D 491/147; A61K 31/4355

(57) ABSTRACT

A process for isolating amorphous berberine with enhanced bioavailability which includes grinding crystalline berberine into amorphous berberine particles having an average size between 100 nm and 200 nm. The grinding step may be accomplished using a horizontal wet bead mill. Alternatively, the crystalline berberine may be ground into amorphous berberine particles having an average size between 125 nm and 175 nm or between 100 nm and 150 nm. The process may further include a step of confirming that all crystalline berberine was converted to amorphous berberine through x-ray diffraction.

### CROSS-REFERENCE TO RELATED APPLICATIONS

The present disclosure is based on and claims priority to U.S. Provisional Patent Application No. 63/640,054 filed Apr. 29, 2024, the disclosure of which is incorporated herein by reference.

### FIELD

The present disclosure relates to nutritional supplements, particularly to berberine nutritional supplements, ingredients, and methods for producing the same.

### BACKGROUND OF THE INVENTION

Berberine HCl (“Berberine”) is a natural alkaloid that has been used for thousands of years for various health benefits including glycemic control, immune function, and other indications. Berberine exists in nature as a crystalline solid, which inherently gives it a low affinity for water and absorption in the body, making it less bioavailable. Berberine has low bioavailability (<1%) which requires it to be dosed multiple times per day at high dosages (e.g. 500 mg 2-3 times daily). Due to its poor bioavailability, 99% of berberine's dose goes into the lower bowel unabsorbed where it ferments by lower gut microbes that produce gas, bloating and abdominal pain. Many end users may experience side effects at these dosages, including G.I. distress, cramps, diarrhea, and the like.

A total of 16 metabolites of berberine have been identified, consisting of ten phase I metabolites, and six phase II metabolites. Xu, P.; Xu, C.; Li, X.; Li, D.; Li, Y.; Jiang, J.; Yang, P.; Duan, G. Rapid identification of berberine metabolites in rat plasma by UHPLC-Q-TOF-MS. Molecules 2019, 24, 1994. Among these metabolites, berberrubine, thalifendine, demethyleneberberine, and jatrorrhizine showed biological activities for antioxidative, hepatoprotective, and hypolipidemic effects.

Studies have shown that when berberine is converted from its crystalline form to its amorphous form, there is an increase in bioavailability of the berberine. There are significant problems, however, with converting berberine to the amorphous configuration. First, if all of the berberine is not converted to the amorphous configuration (i.e. some crystalline berberine remains), then any amorphous berberine will ultimately revert to the crystalline form. Next, while hot melting a substance is a preferred method for conversion of crystalline to amorphous, berberine cannot be converted in this matter due to degradation of the berberine molecule from the heat needed to melt the crystalline structure. Additionally, the use of absorption enhancers or synthetic chemical modification of the berberine molecules to a more absorbable form, changes the active ingredient creating approval and labeling issues and may introduce toxic or harmful solvents into the final product. Moreover, precipitating pure amorphous berberine without any chemical modification or latent crystalline berberine is procedurally and technically difficult.

Applicants are not aware of prior art that demonstrates increased berberine bioavailability by converting berberine from its crystalline to a stable amorphous form without the use of absorption enhancers or chemically modifying the berberine molecules.

M. Zhaojie et al. in “Amorphous Solid Dispersion of Berberine with Absorption Enhancer Demonstrates a Remarkable Hypoglycemic Effect via Improving its Bioavailability” published in the International Journal of Pharmaceutics in 2014 conducted pharmacokinetic studies that demonstrated a 5-fold increase in vivo bioavailability compared to standard berberine by creating an amorphous solid dispersion of berberine with sodium caprate. Zhaojie et al. used the combination of the solvent evaporation method to achieve berberine amorphism as well as the intestinal absorption enhancing properties of sodium caprate to promote absorption. There is no evidence, however, presented by Zhaojie et al. that the resulting berberine created using the solvent evaporation method used is or remains amorphous, e.g. no x-ray diffraction scan is provided. Moreover, research from other studies show the intestinal absorption properties of sodium caprate alone significantly improve crystalline berberine bioavailability. Finally, the augmentation of bioavailability from sodium caprate is in itself problematic because sodium caprate's mechanism of action for enhancing bioavailability does not discriminate between berberine and xenobiotic compounds (with some potentially being unsafe with greater absorption). It therefore has the potential to enhance the performance and toxicity of other xenobiotics that could be used simultaneous with the product thereby unwittingly causing the consumer to be exposed to increased health safety risk.

A commercially available product, Berbevis® produced by Indena S.p.A. (Milan, IT), is formulated with berberine hydrochloride, lecithin, pea protein and grapeseed extract. This product was found to have 9.6 times more bioavailability than standard berberine. Indena's product makes use of a proprietary formulation called Phytosome that works by improving the hydrophilicity of poorly bioavailable actives like berberine. Berbevis uses a food-grade mix of ingredients, e.g., lecithin, pea proteins and a standardized grape seed extract that collectively alter the lipophile/hydrophile balance to make the predominately hydrophobic crystalline berberine become more water dispersible and hence, more readily absorbable. Of the berberine approaches documented to have improved bioavailability, Berbevis has the highest bioavailability at 9.6% improved vs unaltered berberine.

A Chinese company, Nanjing Nutrabuilding Bio-tech Co., LTD. (Nanjing, China) synthetically prepared a precursor of berberine called dihydroberberine (dhBB) for its commercially available product Gluco Vantage. See, U.S. Pat. Nos. 10,278,961 and 11,903,932. When orally dosed, dhBB is absorbed in the intestine then is converted in the blood to berberine. Since dhBB passes into the blood more readily before conversion to berberine, this increases the bioavailability of berberine better than if berberine alone was orally dosed. The oral dosage of dhBB is reported to have a 5 times higher bioavailability of berberine versus an oral dosage of standard berberine.

Accordingly, there is a need for the production of stable amorphous berberine without causing a chemical change to the berberine molecules or adding potentially harmful xenobiotics and a method for accomplishing the same. There is a further need for the production of stable amorphous berberine as a supplement for increased bioavailability to consumers.

### SUMMARY OF THE INVENTION

The research team has identified a solution for increasing berberine's bioavailability by converting berberine from its native crystalline form to an amorphous form by reducing the particle size of the standard berberine material and stabilizing and isolating the amorphous berberine with a lipophilic or amphiphilic excipient.

Accordingly, the present application discloses a process for isolating amorphous berberine with enhanced bioavailability. The process includes grinding crystalline berberine into amorphous berberine particles having an average size between 100 nm and 200 nm. In certain embodiments, the grinding step is accomplished using a horizontal wet bead mill. Alternatively, the crystalline berberine may be ground into amorphous berberine particles having an average size between 125 nm and 175 nm or between 100 nm and 150 nm. The process may further include a step of confirming that all crystalline berberine was converted to amorphous berberine through x-ray diffraction.

The present application also discloses a process for producing stable amorphous berberine particles. This process grinds crystalline berberine into amorphous berberine particles having an average size between 100 nm and 200 nm and confirms that all crystalline berberine was converted to amorphous berberine through x-ray diffraction. The process used typically involves combining crystalline berberine with a lipophilic or amphiphilic excipient in an aqueous slurry and milling the combination in a horizontal wet bead mill. This results in coating the amorphous berberine with the lipophilic or amphiphilic excipient. The resulting milled slurry is discharged from the mill, after which water is removed by drying (e.g., a spray dryer). Any agglomerated powder particles in the dry powder are broken up using any number of mechanical means/screening. The crystalline berberine may be ground into amorphous berberine particles having an average size between 125 nm and 175 nm or between 100 nm and 150 nm. The lipophilic excipient may be selected from one of: PEG-40 hydrogenated castor oil, PEG-60 Hydrogenated castor oil, polyoxyl 35 hydrogenated castor oil, polyoxyethylene (20) sorbitan monolaurate and polyoxyethylene sorbitan monooleate, glyceryl monooleate, caprylocaproyl macrogol-8 glycerides, oleoyl polyoxyl-6 glycerides, linoleoyl polyoxyl-6 glycerides, amylopectin and amylose. The amphiphilic excipient may be selected from one of: methylcellulose (MC), ethylcellulose (EC), hydroxypropylcellulose (HPC), hydroxyethylcellulose (HEC), hydroxypropylmethylcellulose (HPMC), cellulose acetate butyrate (CAB), hydroxypropylmethylcellulose acetyl succinate (HPMCAS), cellulose acetate adipate (CA AdP), cellulose acetate suberate (CA Sub); monochloroacetic acid (MCAd), cellulose acetate butyrate sebacate (CAB Seb), cellulose acetate phthalate suberate (CAP Sub), cellulose acetate phthalate sebacate (CAP Seb), cellulose acetate phthalate suberate (CAB Sub), cellulose acetate sebacate (CA Seb), cellulose acetate phthalate adipate (CAB Adp), cellulose acetate phthalate adipate (CAP Adp), and cellulose propionate adipate (CP Adp).

A stable amorphous berberine product having increased bioavailability is also disclosed. The stable amorphous berberine product has an amorphous berberine particle having an average particle size between 100 nm and 200 nm and a lipophilic or amphiphilic excipient coating surrounding the amorphous berberine particle. Again, the lipophilic excipient may be selected from one of: PEG-40 hydrogenated castor oil, PEG-60 Hydrogenated castor oil, polyoxyl 35 hydrogenated castor oil, polyoxyethylene (20) sorbitan monolaurate and polyoxyethylene sorbitan monooleate, glyceryl monooleate, caprylocaproyl macrogol-8 glycerides, oleoyl polyoxyl-6 glycerides, linoleoyl polyoxyl-6 glycerides, amylopectin and amylose. Again, the amphiphilic excipient may be selected from one of methylcellulose (MC), ethylcellulose (EC), hydroxypropylcellulose (HPC), hydroxyethylcellulose (HEC), hydroxypropylmethylcellulose (HPMC), cellulose acetate butyrate (CAB), hydroxypropylmethylcellulose acetyl succinate (HPMCAS), cellulose acetate adipate (CA AdP), cellulose acetate suberate (CA Sub), monochloroacetic acid (MCAd), cellulose acetate butyrate sebacate (CAB Seb), cellulose acetate phthalate suberate (CAP Sub), cellulose acetate phthalate sebacate (CAP Seb), cellulose acetate phthalate suberate (CAB Sub), cellulose acetate sebacate (CA Seb), cellulose acetate phthalate adipate (CAB Adp), cellulose acetate phthalate adipate (CAP Adp), and cellulose propionate adipate (CP Adp).

A process for manufacturing a stable amorphous berberine supplement is disclosed. This process includes grinding crystalline berberine into amorphous berberine particles having an average size between 100 nm and 200 nm and then confirming that all crystalline berberine was converted to amorphous berberine through x-ray diffraction. The process involves combining crystalline berberine with a lipophilic or amphiphilic excipient in an aqueous slurry and milling the combination in a horizontal wet bead mill. This results in coating the amorphous berberine with the lipophilic or amphiphilic excipient. The resulting milled slurry is discharged from the mill, after which water is removed by drying (e.g., a spray dryer). Any agglomerated powder particles in the dry powder are broken up using any number of mechanical means/screening. The coated amorphous berberine product is packaged into one of: a capsule, a tablet, a patch, a suppository, a cream, a powder, or a liquid mixture. Again, the lipophilic excipient may be selected from one of: PEG-40 hydrogenated castor oil, PEG-60 Hydrogenated castor oil, polyoxyl 35 hydrogenated castor oil, polyoxyethylene (20) sorbitan monolaurate and polyoxyethylene sorbitan monooleate, glyceryl monooleate, caprylocaproyl macrogol-8 glycerides, oleoyl polyoxyl-6 glycerides, linoleoyl polyoxyl-6 glycerides, amylopectin and amylose. Again, the amphiphilic excipient may be selected from one of methylcellulose (MC), ethylcellulose (EC), hydroxypropylcellulose (HPC), hydroxyethylcellulose (HEC), hydroxypropylmethylcellulose (HPMC), cellulose acetate butyrate (CAB), hydroxypropylmethylcellulose acetyl succinate (HPMCAS), cellulose acetate adipate (CA AdP), cellulose acetate suberate (CA Sub), monochloroacetic acid (MCAd), cellulose acetate butyrate sebacate (CAB Seb), cellulose acetate phthalate suberate (CAP Sub), cellulose acetate phthalate sebacate (CAP Seb), cellulose acetate phthalate suberate (CAB Sub), cellulose acetate sebacate (CA Seb), cellulose acetate phthalate adipate (CAB Adp), cellulose acetate phthalate adipate (CAP Adp), and cellulose propionate adipate (CP Adp).

An amorphous berberine supplement is disclosed that includes a stable amorphous berberine product having increased bioavailability. The supplement includes amorphous berberine particles having an average particle size between 100 nm and 200 nm and a lipophilic or amphiphilic excipient coating surrounding the amorphous berberine particles. Alternatively, the crystalline berberine may be ground into amorphous berberine particles having an average size between 125 nm and 175 nm or between 100 nm and 150 nm. The coated particles are then formed into one of: a capsule, a tablet, a patch, a suppository, a cream, a powder, or a liquid mixture. The lipophilic excipient is selected from one of: PEG-40 hydrogenated castor oil, PEG-60 Hydrogenated castor oil, polyoxyl 35 hydrogenated castor oil, polyoxyethylene (20) sorbitan monolaurate and polyoxyethylene sorbitan monooleate, glyceryl monooleate, caprylocaproyl macrogol-8 glycerides, oleoyl polyoxyl-6 glycerides, linoleoyl polyoxyl-6 glycerides, amylopectin and amylose. The amphiphilic excipient may be selected from one of methylcellulose (MC), ethylcellulose (EC), hydroxypropylcellulose (HPC), hydroxyethylcellulose (HEC), hydroxypropylmethylcellulose (HPMC), cellulose acetate butyrate (CAB), hydroxypropylmethylcellulose acetyl succinate (HPMCAS), cellulose acetate adipate (CA AdP), cellulose acetate suberate (CA Sub), monochloroacetic acid (MCAd), cellulose acetate butyrate sebacate (CAB Seb), cellulose acetate phthalate suberate (CAP Sub), cellulose acetate phthalate sebacate (CAP Seb), cellulose acetate phthalate suberate (CAB Sub), cellulose acetate sebacate (CA Seb), cellulose acetate phthalate adipate (CAB Adp), cellulose acetate phthalate adipate (CAP Adp), and cellulose propionate adipate (CP Adp).

### DETAILED DESCRIPTION OF THE INVENTION

A crystalline solid has a long range of ordered molecules and a sharp melting point. In contrast, an amorphous compound has a short range of ordered molecules and an irregular arrangement of its atoms. Amorphous solids are non-crystalline substances that do not possess a characteristic geometrical arrangement. They also do not have a fixed melting point. Despite their name, amorphous materials do display a somewhat orderly arrangement of atoms that may extend up to a few Angstrom units. Amorphous solids lack distinct edges and are therefore non-crystalline.

Berberine is an isoquinoline alkaloid isolated from Coptidis Rhizoma. Berberine occurs in nature as a crystalline solid. Berbeine's amorphous form is unstable and continuously desires to re-crystalize. Re-crystallization of berberine readily occurs when the amorphous form contacts any crystalline form. Accordingly, it is imperative in creating amorphous berberine to be able to stabilize the amorphous form so that it does not re-crystalize.

The present application is directed to a novel process for creating, isolating and stabilizing amorphous berberine to increase the bioavailability without chemically altering chemical structure of berberine, and the resultant berberine product.

The process involves grinding naturally occurring crystalline berberine to an average particle size between 100 and 200 nanometers (nm), preferably to an average size between 125 and 175 nm, and most preferably to an average size between 100 nm and 150 nm using a horizontal bead mill. The resultant berberine is then tested by x-ray diffraction to ensure that 100% of the berberine is converted to the amorphous form. The process used typically involves combining crystalline berberine with a lipophilic or amphiphilic excipient in an aqueous slurry and milling the combination in a horizontal wet bead mill. This results in coating the amorphous berberine with the lipophilic or amphiphilic excipient. The resulting milled slurry is discharged from the mill, after which water is removed by drying (e.g., a spray dryer). Any agglomerated powder particles in the dry powder are broken up using any number of mechanical means/screening

Lipid excipients are mainly categorized as triglycerides (TG), mixed glycerides and polar oils, water-soluble and insoluble surfactants, co-solvents, and other additives. In one embodiment, the lipophilic excipient is selected from one of: PEG-40 hydrogenated castor oil, PEG-60 Hydrogenated castor oil, polyoxyl 35 hydrogenated castor oil, polyoxyethylene (20) sorbitan monolaurate and polyoxyethylene sorbitan monooleate, glyceryl monooleate, caprylocaproyl macrogol-8 glycerides, oleoyl polyoxyl-6 glycerides, linoleoyl polyoxyl-6 glycerides, amylopectin and amylose.

Amphiphilic excipients possess both hydrophilic and lipophilic properties. Applicable amphiphilic excipients include pH-responsive cellulose esters and ethers such as methylcellulose (MC), ethylcellulose (EC), hydroxypropylcellulose (HPC), hydroxyethylcellulose (HEC), hydroxypropylmethylcellulose (HPMC), cellulose acetate butyrate (CAB), and hydroxypropylmethylcellulose acetyl succinate (HPMCAS); or Cellulose @-carboxy esters such as cellulose acetate adipate (CA AdP), cellulose acetate suberate (CA Sub), monochloroacetic acid (MCAd), cellulose acetate butyrate sebacate (CAB Seb), cellulose acetate phthalate suberate (CAP Sub), cellulose acetate phthalate sebacate (CAP Seb), cellulose acetate phthalate suberate (CAB Sub), cellulose acetate sebacate (CA Seb), cellulose acetate phthalate adipate (CAB Adp), cellulose acetate phthalate adipate (CAP Adp), and cellulose propionate adipate (CP Adp).

The resultant amorphous berberine product has enhanced bioavailability, at least 10x, compared to standard crystalline berberine. The increased bioavailability allows the dose of the product to be lower than other known products and permits the required number of doses to be lower to achieve the desired benefits, without detrimental side-effects. Further, due to the addition of the lipophilic or amphiphilic excipient, the stability of the amorphous berberine is significantly enhanced. Moreover, this is all achieved without the use of absorption enhancers or synthetic chemical modification of the berberine molecules to a more absorbable form.

The stable amorphous berberine, when coated with the lipophilic or amphiphilic excipient, becomes a stable amorphous berberine product having increased bioavailability. This amorphous berberine product having increased bioavailability may be manufactured into a supplement by packaging the coated amorphous berberine product into one of: a capsule, a tablet, a patch, a suppository, or a cream. Alternatively, the amorphous berberine product having increased bioavailability may be distributed in powder from or in a liquid mixture.

1. A process for isolating amorphous berberine with enhanced bioavailability, comprising the step of grinding crystalline berberine into amorphous berberine particles having an average size between 100 nm and 200 nm.

2. The process of claim 1 wherein the step of grinding comprises grinding with a horizontal wet bead mill, wherein the step of coating is accomplished by adding the lipophilic or amphiphilic excipient to the horizontal bead mill during the step of grinding to create coated amorphous berberine particles, and wherein the process further comprises a step of: drying the coated amorphous berberine particles.

3. The process of claim 1 wherein the step of grinding comprises grinding crystalline berberine into amorphous berberine particles having an average size between 125 nm and 175 nm.

4. The process of claim 1 wherein the step of grinding comprises grinding crystalline berberine into amorphous berberine particles having an average size between 100 nm and 150 nm.

5. The process of claim 1 or 2 further comprising the step of: confirming that all crystalline berberine was converted to amorphous berberine through x-ray diffraction.

6. A process for producing stable amorphous berberine particles, the process comprising the steps of: grinding crystalline berberine into amorphous berberine particles having an average size between 100 nm and 200 nm; confirming that all crystalline berberine was converted to amorphous berberine through x-ray diffraction; and coating the amorphous berberine with a lipophilic or amphiphilic excipient.

7. The process of claim 6 wherein the step of grinding comprises grinding with a horizontal wet bead mill, the step of coating comprises adding the lipophilic or amphiphilic excipient to the horizontal bead mill during the step of grinding to create coated amorphous berberine particles, and wherein the process further comprises a step of drying the coated amorphous berberine particles.

8. The process of claim 6 wherein the step of grinding comprises grinding crystalline berberine into amorphous berberine particles having an average size between 125 nm and 175 nm.

9. The process of claim 6 wherein the step of grinding comprises grinding crystalline berberine into amorphous berberine particles having an average size between 100 nm and 150 nm.

10. The process of claim 6 wherein the lipophilic excipient is selected from one of: PEG-40 hydrogenated castor oil, PEG-60 Hydrogenated castor oil, polyoxyl 35 hydrogenated castor oil, polyoxyethylene (20) sorbitan monolaurate and polyoxyethylene sorbitan monooleate, glyceryl monooleate, caprylocaproyl macrogol-8 glycerides, oleoyl polyoxyl-6 glycerides, linoleoyl polyoxyl-6 glycerides, amylopectin and amylose.

11. The process of claim 6 wherein the amphiphilic excipient is selected from one of: methylcellulose (MC), ethylcellulose (EC), hydroxypropylcellulose (HPC), hydroxyethylcellulose (HEC), hydroxypropylmethylcellulose (HPMC), cellulose acetate butyrate (CAB), hydroxypropylmethylcellulose acetyl succinate (HPMCAS), cellulose acetate adipate (CA AdP), cellulose acetate suberate (CA Sub), monochloroacetic acid (MCAd), cellulose acetate butyrate sebacate (CAB Seb), cellulose acetate phthalate suberate (CAP Sub), cellulose acetate phthalate sebacate (CAP Seb), cellulose acetate phthalate suberate (CAB Sub), cellulose acetate sebacate (CA Seb), cellulose acetate phthalate adipate (CAB Adp), cellulose acetate phthalate adipate (CAP Adp), and cellulose propionate adipate (CP Adp).

12. A stable amorphous berberine product having increased bioavailability comprising: an amorphous berberine particle having an average particle size between 100 nm and 200 nm and a coating surrounding the amorphous berberine particle, the coating comprising a lipophilic or amphiphilic excipient.

13. The stable amorphous berberine product of claim 12, wherein the lipophilic excipient is selected from one of: PEG-40 hydrogenated castor oil, PEG-60 Hydrogenated castor oil, polyoxyl 35 hydrogenated castor oil, polyoxyethylene (20) sorbitan monolaurate and polyoxyethylene sorbitan monooleate, glyceryl monooleate, caprylocaproyl macrogol-8 glycerides, oleoyl polyoxyl-6 glycerides, linoleoyl polyoxyl-6 glycerides, amylopectin and amylose.

14. The stable amorphous berberine product of claim 12, wherein the amphiphilic excipient is selected from one of: methylcellulose (MC), ethylcellulose (EC), hydroxypropylcellulose (HPC), hydroxyethylcellulose (HEC), hydroxypropylmethylcellulose (HPMC), cellulose acetate butyrate (CAB), hydroxypropylmethylcellulose acetyl succinate (HPMCAS), cellulose acetate adipate (CA AdP), cellulose acetate suberate (CA Sub), monochloroacetic acid (MCAd), cellulose acetate butyrate sebacate (CAB Seb), cellulose acetate phthalate suberate (CAP Sub), cellulose acetate phthalate sebacate (CAP Seb), cellulose acetate phthalate suberate (CAB Sub), cellulose acetate sebacate (CA Seb), cellulose acetate phthalate adipate (CAB Adp), cellulose acetate phthalate adipate (CAP Adp), and cellulose propionate adipate (CP Adp).

15. A process for manufacturing a stable amorphous berberine supplement, the process comprising: grinding crystalline berberine into amorphous berberine particles having a size between 100 nm and 200 nm; confirming that all crystalline berberine was converted to amorphous berberine through x-ray diffraction; coating the amorphous berberine with a lipophilic or amphiphilic excipient to create a coated amorphous berberine product; drying the coated amorphous berberine product; and packaging the amorphous berberine product into one of: a capsule, a tablet, a patch, a suppository, a cream, a powder, or a liquid mixture.

16. The process of claim 15, wherein the lipophilic excipient is selected from one of: PEG-40 hydrogenated castor oil, PEG-60 Hydrogenated castor oil, polyoxyl 35 hydrogenated castor oil, polyoxyethylene (20) sorbitan monolaurate and polyoxyethylene sorbitan monooleate, glyceryl monooleate, caprylocaproyl macrogol-8 glycerides, oleoyl polyoxyl-6 glycerides, linoleoyl polyoxyl-6 glycerides, amylopectin and amylose.

17. The process of claim 15, wherein the amphiphilic excipient is selected from one of: methylcellulose (MC), ethylcellulose (EC), hydroxypropylcellulose (HPC), hydroxyethylcellulose (HEC), hydroxypropylmethylcellulose (HPMC), cellulose acetate butyrate (CAB), hydroxypropylmethylcellulose acetyl succinate (HPMCAS), cellulose acetate adipate (CA AdP), cellulose acetate suberate (CA Sub), monochloroacetic acid (MCAd), cellulose acetate butyrate sebacate (CAB Seb), cellulose acetate phthalate suberate (CAP Sub), cellulose acetate phthalate sebacate (CAP Seb), cellulose acetate phthalate suberate (CAB Sub), cellulose acetate sebacate (CA Seb), cellulose acetate phthalate adipate (CAB Adp), cellulose acetate phthalate adipate (CAP Adp), and cellulose propionate adipate (CP Adp).

18. An amorphous berberine supplement comprising a stable amorphous berberine product having increased bioavailability comprising: amorphous berberine particles having an average particle size between 100 nm and 200 nm and a coating surrounding the amorphous berberine particles, the coating comprising a lipophilic or amphiphilic excipient, wherein coated particles are formed into one of: a capsule, a tablet, a patch, a suppository, a cream, a powder, or a liquid mixture.

19. The process of claim 18, wherein the lipophilic excipient is selected from one of: PEG-40 hydrogenated castor oil, PEG-60 Hydrogenated castor oil, polyoxyl 35 hydrogenated castor oil, polyoxyethylene (20) sorbitan monolaurate and polyoxyethylene sorbitan monooleate, glyceryl monooleate, caprylocaproyl macrogol-8 glycerides, oleoyl polyoxyl-6 glycerides, linoleoyl polyoxyl-6 glycerides, amylopectin and amylose.

20. The process of claim 18, wherein the amphiphilic excipient is selected from one of: methylcellulose (MC), ethylcellulose (EC), hydroxypropylcellulose (HPC), hydroxyethylcellulose (HEC), hydroxypropylmethylcellulose (HPMC), cellulose acetate butyrate (CAB), hydroxypropylmethylcellulose acetyl succinate (HPMCAS), cellulose acetate adipate (CA AdP), cellulose acetate suberate (CA Sub), monochloroacetic acid (MCAd), cellulose acetate butyrate sebacate (CAB Seb), cellulose acetate phthalate suberate (CAP Sub), cellulose acetate phthalate sebacate (CAP Seb), cellulose acetate phthalate suberate (CAB Sub), cellulose acetate sebacate (CA Seb), cellulose acetate phthalate adipate (CAB Adp), cellulose acetate phthalate adipate (CAP Adp), and cellulose propionate adipate (CP Adp).

21. The process of claim 18, wherein amorphous berberine particles having an average particle size between 125 nm and 175 nm.

22. The process of claim 18, wherein amorphous berberine particles having an average particle size between 100 nm and 150 nm.
